(19) United States
(12) Patent Application Publication
(10) Pub. No.: US 2022/0168159 A1
(43) Pub. Date: Jun. 2, 2022

(54) ABSORBENT PADS AND GARMENTS

(71) Applicant: JAIMEX NOMINEES PTY LTD, Scoresby, Victoria (AU)

(72) Inventors: Peter Julian Triado, Scoresby, Victoria (AU); Steven Myers, Scoresby, Victoria (AU); Gavin Jayasekere, Scoresby, Victoria (AU)

(21) Appl. No.: 17/613,780

(22) PCT Filed: May 21, 2020
(86) PCT No.: PCT/AU2020/050498

§ 371 Date: Nov. 23, 2021

(30) Foreign Application Priority Data
May 24, 2019 (AU) 2019901784

(51) Int. Cl. A61F 13/74; A61F 13/539; A61F 13/537; A61F 13/14

(52) U.S. Cl. CPC A61F 13/74; A61F 13/539; A61F 2013/15487; A61F 13/141; A61F 13/537

(57) ABSTRACT

A pad for a garment includes a body and capping. The body has a front, a rear and a periphery and includes one or more absorbent layers for absorbing liquid and a gas-permeable barrier overlying the one or more absorbent layers to block the liquid. The capping runs along and caps at least a portion of the periphery to block the liquid. The capping has a front portion overlying the front, a rear portion overlying the rear, and a mounting portion, projecting away from the body, for mounting the pad.

### FIELD OF THE INVENTION

The invention in its various aspects relates to absorbent pads and garments, and to components and methods therefor.

### BACKGROUND

Humans routinely emit various liquids unintentionally, for example through sweating, bladder leakage, menstruation or lactation. Such uncontrolled emissions can be uncomfortable, inconvenient and embarrassing.

Various absorbent pads and garments comprising such pads have been proposed. It is desirable that such pads be comfortable and resistant to leakage.

The present inventors have recognised that the periphery of absorbent pads can be a source of discomfort and/or a point of leakage. With the foregoing in mind, the present invention aims to provide improvements in and for the control of bodily fluids or at least to provide a useful alternative for those concerned with such matters.

### SUMMARY

One aspect of the invention provides a pad, for a garment, comprising

a body having a front, a rear and a periphery and comprising
one or more absorbent layers for absorbing liquid; and
a gas-permeable barrier to block the liquid; and
   capping running along, and capping, at least a portion of the periphery to block the liquid;

the capping comprising
a front portion overlying the front;
a rear portion overlying the rear; and
a mounting portion, projecting away from the body, for mounting the pad.

Optionally the capping runs along, and caps, substantially all of the periphery. Preferably the capping comprises at least one capping element defining at least part of each of the front portion, the rear portion and the mounting portion. The at least one capping element may be one capping element defining the front portion, the rear portion and the mounting portion. Optionally a plurality of the capping elements are distributed about the periphery. The mounting portion is preferably at least 2 mm, or more preferably at least 3 mm, wide. The mounting portion is preferably not more than 25 mm, or more preferably not more than 15 mm, wide.

Optionally the capping is fabric capping. Preferably the capping comprises at least one of polyamide and polyurethane. The capping may be treated with water repellant.

Optionally the pad comprises stitching, e.g. monofilament stitching, fastening at least one of the one or more absorbent layers to at least one other layer of the body, the front portion and the rear portion overlying the stitching.

Each of the one or more absorbent layers may comprise fabric. Each of the one or more absorbent layers may comprise knitted fabric. The pad may comprise two or more of the absorbent layers, in which case preferably two of the absorbent layers are formed of the same material as each other. Preferably the one or more absorbent layers together have weight in range of 700 to 900 GSM.

The body may comprise a wicking layer for wicking liquid from a wearer to the one or more absorbent layers.

Optionally the barrier comprises fluorocarbon nanoparticles and/or polyurethane.

Preferably the barrier is in the range of 0.01 mm to 0.03 mm thick (inclusive).

The pad may comprise silver ions to control odour.

Another aspect of the invention provides a garment comprising the pad mounted via the mounting portion, e.g. mounted by stitching the mounting portion.

Another aspect of the invention provides a pad, for a garment, comprising

a body having a front, a rear and a periphery and comprising
one or more absorbent layers for absorbing liquid;
a gas-permeable barrier overlying the one or more absorbent layers to block the liquid; and

fabric capping running along, and capping, at least a portion of the periphery to block the liquid;

the capping comprising
a front portion overlying the front;
a rear portion overlying the rear.

Another aspect of the invention provides an absorbent pad for an undergarment, comprising

a body having a front, a rear and a periphery and comprising
one or more absorbent layers for absorbing liquid; and
a gas-permeable barrier to block the liquid; and

capping running along, and capping, at least a portion of the periphery to block the liquid;

the capping comprising
a front portion overlying the front;
a rear portion overlying the rear; and

a mounting portion, projecting away from the body, said mounting portion providing a mounting region distinct from the body at where the pad can be affixed to the undergarment.

Another aspect of the invention provides an undergarment comprising a fabric body and carrying an absorbent pad, said absorbent pad comprising

a body having a front, a rear and a periphery and comprising
one or more absorbent layers for absorbing liquid; and
a gas-permeable barrier to block the liquid; and

capping running along, and capping, at least a portion of the periphery to block the liquid;

the capping comprising
a front portion overlying the front;
a rear portion overlying the rear; and

a mounting portion, projecting away from the body, and providing a mounting region distinct from the body;

wherein said pad is affixed to the fabric body of the undergarment by stitching, fusion or adhesion at the mounting region.

The options listed in relation to the first aspect of the invention apply to the further aspects of the invention.

### BRIEF DESCRIPTION OF DRAWINGS

FIG. 1 is a cross-section view of an edge of a pad;

FIG. 2 is a cross-section view of the body and capping of the pad of FIG. 1;

FIG. 3 is a front view of a bra; and

FIG. 4 is a front view of a pair of underpants.

### DETAILED DESCRIPTION

The pad 1 comprises a body 3 and capping 5. Preferably the pad is in the range of 3 mm to 10 mm (inclusive), or more preferably 3 mm to 5 mm (inclusive), thick. The pad 1 is configured to absorb an initial discharge of 15 mL and a slow discharge of 40 mL. A smaller pad configured to absorb a slow discharge of 30 mL is also contemplated.

In this example, the body 3 has a 4-layer construction comprising an inner layer 3a, a middle layer 3b, an outer layer 3c and a barrier 3d.

The inner layer 3a is a wicking layer to sit against (or at least in proximity to) the wearer's body. The inner layer serves to wick moisture away from the wearer towards the other layers of the body 3. The inner layer is preferably thinner than the combined thickness of the absorbent materials of the pad, that is thinner than the combined thickness of layers 3b, 3c in this case. Preferably the layer 3a is thinner than each of the layers 3b, 3c. Most preferably it has a weight in the vicinity of 250-350 GSM. The layer 3a is preferably knitted and is most preferably formed of polyester, e.g. 100% polyester.

The layers 3b, 3c are absorbent layers and are preferably formed of the same material. The material is preferably cotton rich, e.g. 86% cotton and 14% polyester French terry fabric. The material is preferably knitted. Double knitted fabric, such as SCUBA fabric, is another option. Each of the layers 3b, 3c has a weight in the range of 350-450 GSM. Polyester and cotton rich fabric are not the only options. Any one or more of the layers 3a, 3b, 3c may be made up of one or more other materials. Nylon, polypropylene (synthetic), cotton, linen, modal (natural), rayon, deo cell (cellulose material such as viscose, bamboo and rayon) are other options.

One or more of the layers 3a, 3b, 3c preferably have hydrophilic and/or quick-dry finishes. For example, both finishes may be added into dye baths and the fabric is sent through these baths in order for the treatments to be absorbed by the fibers in the fabric. The fabric is then sent through rollers to squeeze out the fluid, and dried and finished so the chemicals are fixed to the fibers. The fabric for the layer 3a may be treated separately to the fabric for the layers 3b, 3c to suit the different properties of these fabrics.

The quick dry finish enhances the rate at which liquid is evaporated, by the wearer's body, to escape via the barrier 3d.

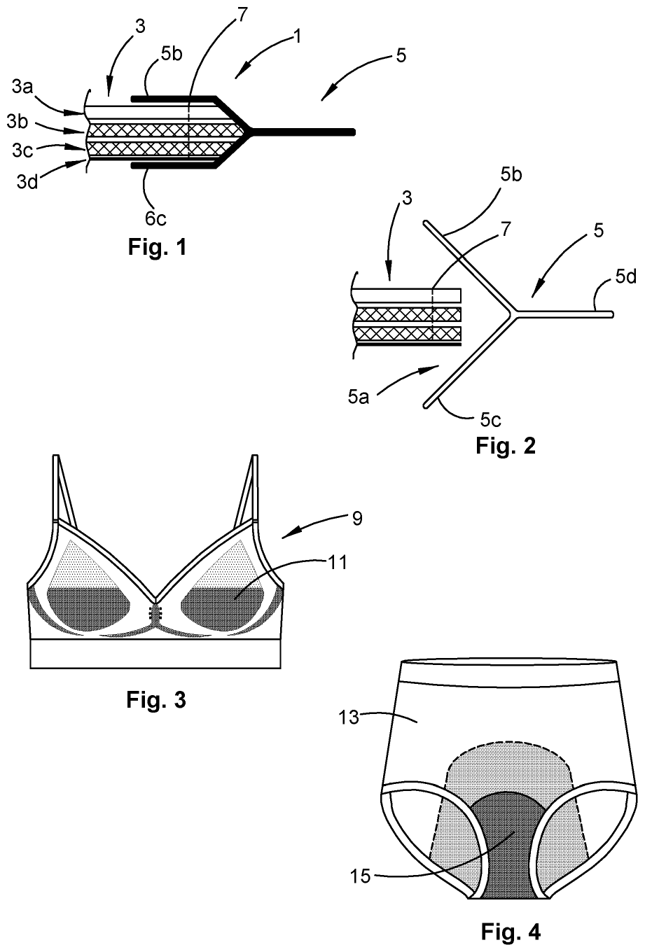

The multilayer construction is preferred. Two layers 3b, 3c are believed to be more effective than a single layer of equivalent thickness. This is believed to be associated with moisture being more inclined to wick laterally throughout the layer 3b rather than to migrate outwardly away from the wearer (corresponding downwards as drawn in FIG. 1).

The layers 3a, 3b, 3c are preferably treated with a silver-based antimicrobial agent (e.g. silver dioxide or silver salt) to reduce odours whereby, in this example, those layers are quick dry, antibacterial and anti-odour layers. The treatment may comprise treatment with antimicrobial nanoparticles and quick dry treatment.

The barrier 3d is a moisture trapping barrier comprising a suitable waterproofing material. Polyurethane laminate or coating in the vicinity of 0.02 mm thick is one option. Fluorocarbon nanoparticles are another option.

The barrier 3d spans substantially all of the layer 3c to block the movement of liquid to retain the absorbed bodily liquids. On the other hand, the barrier 3d is gas permeable to facilitate the evaporation of such liquids.

Stitching 7 keeps the layers of the body 3 together. The stitching 7 runs about the periphery of the body 3. In this example, the stitching is set in the range of 3 mm to 15 mm (inclusive), e.g. 4 mm, inwardly from the edge of the body 3 when that body is in its free-condition as illustrated in FIG. 2.

Monofilament stitching is preferred. Multifilament and multi-ply yarn are other options. Relative to other potential threads, monofilament is less inclined to wick liquid. Preferably this stitching, in registration with the overlying portions of the capping, is the only stitching mutually connecting layers of the body 3.

In the past, absorbent pads have been sealed around the edges with the aid of heat-melt tapes (or similar adhesives) and mounted within clothing by fusing or adhering a face of the pad to a surface of the clothing, and/or via stitching running about a peripheral margin of the pad. The present inventors have recognised that this area is a potential source of discomfort to the wearer of the clothing and/or leakage or reduced performance of the absorbent pad. Following these existing approaches, moisture can wick to, and leak from, the edges of the absorbent material as stitching through the absorbent material can define points of leakage.

Adhesively coated faces of the pad and edge margins can be less breathable and therefore uncomfortable, and can degrade with repeated washing of the garment.

The pad 1 comprises capping 5 running about the periphery of the body 3. In this example, the capping 5 has a Y-shaped profile in its free-condition (FIG. 2) defining a channel 5a into which the periphery of the body 3 is receivable. The three arms of the Y-shaped profile comprise a 10 mm long rear portion 5b (to sit behind the body 3), a 10 mm long front portion 5c (to sit in front of the body 3) and an 8 mm long mounting flange 5d.

The capping 5 is preferably a water-repellant fabric to form a breathable edge seal. The fabric is preferably polyamide or polyurethane treated with a suitable water repellant such as Anthydrin SC. Sandwiching the edge of the body 3 between a pair of plastic tapes would be another option for capping that edge although the one-piece interwoven construction of the capping 5 is preferred in that the end result is more breathable, more flexible and more comfortable to the wearer.

The capping 5 is preferably bonded to the body 3. Preferably heat melt polyurethane adhesive or other bonding agent is applied to the interior of the channel 5a. Bemis™ 3412-00-7 mm×2 is a preferred option. The edge of the body 3 is wedged into the channel 5 and the assembly is pressed and heated to bond the capping 5 to the body 3 in the configuration illustrated in FIG. 1.

The flange 5d is preferably aligned with the body 3d to facilitate mounting, e.g. in the case of a planar body 3d, the flange 5d may be co-planar to the body 3d. Contoured pads (which may, for example, incorporate darts) are also possible. In the case of contoured pads, the flange 5d is preferably an extension of the contoured shape of the pad.

FIG. 5 is a plan view of a gusset pad 15 in its substantially-planar free-condition. The pad comprises the body 3 having a convex front 3ᵢ, a convex rear 3ᵢᵢ and a pair of concave sides.

The periphery of the body 3 is capped by capping 5 made up of four capping pieces. The capping pieces are cut from a continuous run of capping having substantially uniform cross-section along its length. The capping piece 5ᵢ is cut to a length commensurate with the arcuate front 3ᵢ and bonded in place along that edge so that the ends line up with the sides of the body 3 so as to define extensions of those sides. The rear capping 5ᵢᵢ is likewise installed along the rear 3ᵢᵢ.

Side capping pieces 5ᵢᵢᵢ, 5ᵢᵥ are substantially identical to each other. Each is cut to a length corresponding to the length of the extended edges at the sides of the body 3 (that is, cut to a length corresponding to the side length of the assembly 3, 5ᵢ, 5ᵢᵢ). The side capping pieces 5ᵢᵢᵢ, 5ᵢᵥ are positioned and bonded in place along these edges so that the end portions of their valleys embrace the end portions of the pieces 5ᵢ, 5ᵢᵢᵢ to define the overlapping regions 17 (hatched in FIG. 5). This corner treatment seals the corners of the pad.

So installed, the capping 5 defines a substantially continuous peripheral mounting flange. A continuous peripheral mounting portion enables the pad 15 to be fastened to the body of the garment via a substantially continuous closed shape of fastening. Other variants are possible. By way of example, the pad 15 is designed to sit inside the body of a garment. In another variant, the side capping pieces 5ᵢᵢᵢ, 5ᵢᵥ might be substituted by flangeless capping and the remaining front and rear capping pieces 5ᵢ, 5ᵢᵢ stitched into a gap in the garment body, whereby the pad 15 defines the crotch of the garment without an overlying layer of fabric. In yet another variant, the flange portion of each capping piece might itself be discontinuous, e.g. the continuous run of capping from which the pieces might be cut may have tabs spaced along its length in place of the mounting flange 5d.

Whilst there are a range of possibilities, a continuous peripheral mounting region is preferred. Most preferably, the front and back of the pad 15 are stitched into the body of the garment. Cover stitching, as schematically suggested by zigzags 19, covering free edges of the mounting flanges are preferred to secure the edges for comfort and reduced wear of the edges.

FIG. 6 illustrates a preferred mode of fastening the concave sides of the pad 15. Elastic trim 21 and the flange 5d sandwich fabric 23 of the garment body. This layered construction is held together by a line of stitching 25, which in this example takes the form of cover stitching. This stitching appears as two parallel rows of straight stitching on the outside of the garment.

The capping 5 is preferably produced in a single continuous run emerging from a narrow fabric weaving machine as a single interwoven piece, in contrast to (for example) mutually fastening two simple strips to form a Y-piece.

The continuous run of fabric is then dyed to colour and finished with a waterproof coating.

Preferably the capping is (in its free-condition) straight, and is elastically compliant to adopt the curvatures illustrated in FIG. 5, to deform out of plane when stitched into the garment body, and to comfortably move with the wearer in a manner akin to conventional elastic with which the person of skill in the art is familiar.

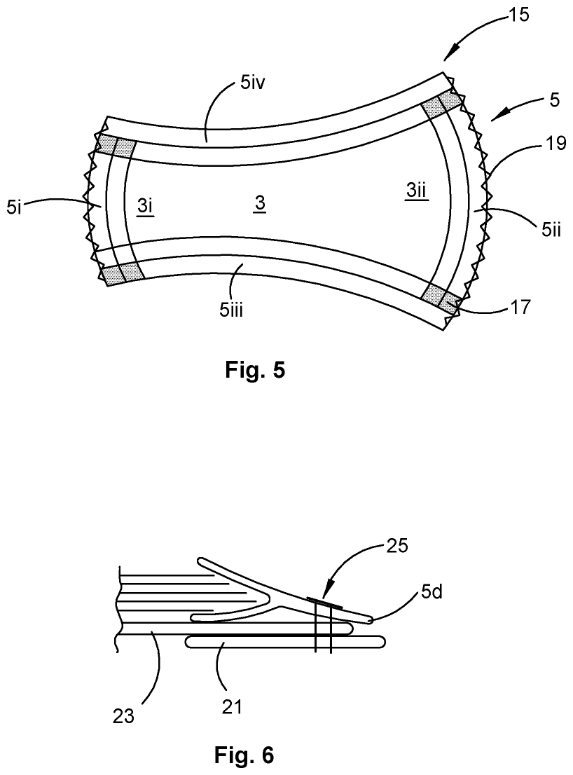

The capping 5 serves two purposes. On the one hand, the periphery of the body 3 is sealed to block leakage from the edges of the body 3 and the stitching 7 is preferably covered so as to block leakage associated with that stitching. On the other hand, the flange 5d is presented to enable the pad to be stitched into the garment without stitching through the absorbent material of the body 3. FIG. 3 illustrates a bra 9 comprising a pair of pads 11 each having a length and width in the range of about 10 cm to about 20 cm (inclusive). FIG. 4 illustrates underpants 13 having a crotch defined by an absorbent pad 15′ akin to the pad 15.

Different garments to suit men and women are contemplated, e.g. the pad might take the form of a gusset pad for women's underwear in which case the pad may be in the range of about 20 cm to about 30 cm (inclusive) in length and in the range of about 5 cm to about 15 cm (inclusive) wide at its narrower point.

Headbands and shirts, such as t-shirts, are other forms of garments in which the pad may be usefully applied.

The fabric of the garment might overlie the barrier 3d. Alternatively, the barrier 3d may define a portion of an exterior of the garment.

Stitching through the mounting flange 5d is one method of permanently mounting the pad into a garment. Temporary mounting is also possible. By way of example the mounting flange 5d and the body of a garment might carry complementary portions of a releasable fastening system, such as complementary halves of a set of press studs or complementary portions of hook and loop fastening.

Other mounting arrangements are possible, e.g. a garment may have a pocket for receiving variations of a pad that may not have a mounting region.

Preferred variants of the pad are machine washable. A separable variant of the pad could be washed separately from a remainder of the garment. It also enables a single pad to be transferred from one garment-body to another.

The invention is not limited to the examples described herein. Rather the invention is defined by the claims.

The term ‘comprises’ and its grammatical variants has a meaning that is determined by the context in which it appears. Accordingly, the term should not be interpreted exhaustively unless the context dictates so.

1. A pad, for a garment, comprising:
a body having a front, a rear and a periphery and comprising:
one or more absorbent layers for absorbing liquid; and
a gas-permeable barrier to block the liquid; and
capping running along, and capping, at least a portion of the periphery to block the liquid;
the capping comprising:
a front portion overlying the front;
a rear portion overlying the rear; and
a mounting portion, projecting away from the body, for mounting the pad.

2. The pad of claim 1 wherein the capping runs along, and caps, substantially all of the periphery.

3. The pad of claim 1 wherein the capping comprises at least one capping element defining at least part of each of the front portion, the rear portion and the mounting portion.

4. The pad of claim 3 wherein the at least one capping element is one capping element defining the front portion, the rear portion and the mounting portion.

5. (canceled)

6. The pad of claim 1 wherein the mounting portion is at least 3 mm wide.

7. The pad of claim 1 wherein the capping is fabric capping.

8. (canceled)

9. The pad of claim 1 wherein the capping is treated with water repellant.

10. The pad of claim 1 comprising stitching fastening at least one of the one or more absorbent layers to at least one other layer of the body; and
the front portion and the rear portion overlying the stitching.

11. The pad of claim 10 wherein the stitching is mono-filament stitching.

12. The pad of claim 1 wherein each of the one or more absorbent layers comprises fabric.

13. The pad of claim 1 wherein each of the one or more absorbent layers comprises knitted fabric.

14. The pad of claim 1 comprising two or more of the absorbent layers.

15. The pad of claim 14 wherein the two of the absorbent layers are formed of the same material as each other.

16. (canceled)

17. The pad of claim 1 wherein the body comprises a wicking layer for wicking liquid from a wearer to the one or more absorbent layers.

18. The pad of claim 1 wherein the barrier comprises fluorocarbon nanoparticles.

19. The pad of claim 1 wherein the barrier comprises polyurethane.

20. (canceled)

21. The pad of claim 1 comprising silver ions to control odour.

22. A garment comprising:
a pad mounted via a mounting portion, the pad comprising:
a body having a front, a rear and a periphery and comprising:
one or more absorbent layers for absorbing liquid; and
a gas-permeable barrier to block the liquid; and
capping running along, and capping, at least a portion of the periphery to block the liquid, the capping comprising:
a front portion overlying the front;
a rear portion overlying the rear; and
the mounting portion projecting away from the body.

23. The garment of claim 22 wherein the pad is mounted to the mounting portion by stitching.

24. A pad, for a garment, comprising:
a body having a front, a rear and a periphery and comprising:
one or more absorbent layers for absorbing liquid; and
a gas-permeable barrier overlying the one or more absorbent layers to block the liquid; and
fabric capping running along, and capping, at least a portion of the periphery to block the liquid;
the capping comprising:
a front portion overlying the front; and
a rear portion overlying the rear.
